(19) United States
(12) Patent Application Publication
(10) Pub. No.: US 2023/0307142 A1
(43) Pub. Date: Sep. 28, 2023

(54) FALL RISK ANALYSIS USING BALANCE PROFILES

(71) Applicant: ZIBRIO. The Balance Company, Houston, TX (US)

(72) Inventors: Katharine Forth, Houston, TX (US); Erez Lieberman Aiden, Houston, TX (US); Kristin Bartlett, Houston, TX (US)

(73) Assignee: ZIBRIO. The Balance Company, Houston, TX (US)

(21) Appl. No.: 18/188,974

(22) Filed: Mar. 23, 2023

Related U.S. Application Data
(60) Provisional application No. 63/269,877, filed on Mar. 24, 2022.

(51) Int. Cl. G16H 50/30; G16H 10/60

(52) U.S. Cl. CPC G16H 50/30; G16H 10/60; G06Q 30/0631

(57) ABSTRACT

Aspects of this disclosure relate to methods and systems for assessing multifactorial balance health and implementation of a risk alert system. The fall risk information can be used to notify the person and/or a third-party monitoring person (e.g., doctor, physical therapist, personal trainer, etc.) of the person's fall risk. This information may be used to monitor and track changes in fall risk that may be impacted by changes in health status, lifestyle behaviors, or medical treatment. Furthermore, the fall risk classification may help individuals be more careful on the days they are more at risk for falling.

### CROSS-REFERENCE TO RELATED PATENT APPLICATIONS

This patent application is related to and claims the benefit of priority of U.S. Provisional Patent Application No. 63/269,877 to Forth et al. filed on Mar. 24, 2022, and entitled “Fall Risk Analysis Using Balance Profiles,” which is incorporated herein by reference.

### BACKGROUND

Unintentional falls account for greater than 30,000 annual deaths within the US population. Seniors are most vulnerable to falling and, as a result, suffer more than 300,000 hip fractures a year. Of those who fracture a hip, 50% will never return to their homes. The poor balance that contributes to these fall events often declines for decades in advance of the fall event, yet the conventional method for tackling poor balance is to seek medical diagnostics and interventions only after a fall has occurred or the patient has a very serious balance problem. In fact, the current best predictor of a fall is whether someone has already fallen.

To truly improve the statistics of falls across the country, preventive intervention should be performed in advance of the first fall. Balance is similar to other physical performances, it can be improved with practice and, conversely, deteriorates with disuse. A number of lifestyle and health factors are known to influence one's balance, such as exercise, strength, sleep, cognitive functioning, vitamin D supplements, and medication management. Lifestyle changes to improve balance will take time to build up their protective effect. Measuring balance and fall risk affords the opportunity to detect subtle balance changes that can occur with health and lifestyle adjustments.

The human balance control system is very complex with three or more sensory inputs creating a repertoire of motor outputs, each with differing strategies that are affected by subconscious and conscious control, experience, context, and personality. The circumstances surrounding falling further complicates matters as the source of a fall can be from numerous intrinsic and extrinsic factors. Consequently, predicting falls with a basic measure of balance is insufficient on its own. The added insight and predictive power that machine learning techniques provide for human balance control systems can facilitate a more accurate prediction of falls. However, there remains a need for improved postural stability representation.

Shortcomings mentioned here are only representative and are included simply to highlight that a need exists for improved information handling systems. Embodiments described herein address certain shortcomings but not necessarily each and every one described here or known in the art. Furthermore, embodiments described herein may present other benefits than, and be used in other applications than, those of the shortcomings described herein.

### SUMMARY

In general, aspects of this disclosure relate to methods and systems for assessing multifactorial balance health and implementation of a risk alert system. The fall risk information can be used to notify the person and/or a third-party monitoring person (e.g., doctor, physical therapist, personal trainer, etc.) of the person's fall risk. This information may be used to monitor and track changes in fall risk that may be impacted by changes in health status, lifestyle behaviors, or medical treatment. Furthermore, the fall risk classification may help individuals be more careful on the days they are more at risk for falling. This contrasts with the general guidelines for preventing falls that are unrealistic in their expectation of increased vigilance and attention at all times. Alerting someone to their fall risk level empowers them to act in the short term, such as to use a cane when the fall risk level is high, or for seeking professional advice for making lifestyle changes for long term improvement of fall risk. In some embodiments, data may be collected over hours, days, weeks and/or months and long-term predictions may be formed for the individual.

According to some embodiments, a method for assessing multifactorial balance health may include determining a balance profile of a patient; determining a fall risk for the patient by executing a trained machine learning algorithm with the balance profile (comprising one or more of postural state analysis, at least one healthcare record, and at least one game report) as an input; and providing a notification to at least one user based on the fall risk.

In some embodiments, providing the notification comprises transmitting a message to the patient. This may include transmitting a notification to an information handling system and/or electronic device associated with the patient, for example. In some embodiments, providing the notification to the at least one user is based on the fall risk satisfying one or more criteria.

According to some embodiments, the balance profile is based on a punctuated equilibrium model (PEM) for the patient. The PEM may be defined as periods of stability punctuated by dynamic trajectories.

According to certain embodiments, determining the fall risk is further based on at least one of health care records, game reports, and/or motion data from an electronic device. The electronic device may be a computer program product or information handling system, and may be associated with the patient and/or a third-party monitoring person.

According to certain embodiments, the method further comprises providing a product referral based on the fall risk satisfying one or more criteria. For example, the method may further comprise suggesting a physical or electronic shopping modality to display products for the patient based on a determined level of health or fall risk. The method may further comprise presenting products via physical or electronic advertising material automatically to the patient based on the fall risk satisfying one or more criteria.

In general, technology described in embodiments herein provides a system and method for determining a person's fall risk and/or composite balance score. The technology may be used, for example, by seniors, athletes, patients, doctors, physical therapists, nurses, astronauts, and/or any person that needs to assess fall risk or postural stability.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF THE DRAWINGS

For a more complete understanding of the disclosed system and methods, reference is now made to the following descriptions taken in conjunction with the accompanying drawings.

FIG. 1 is a block diagram illustrating a system for processing health balance information according to some embodiments of the disclosure.

FIGS. 2A and 2B are flowchart diagrams depicting a method for suggesting a shopping modality based on balance health according to some embodiments of the disclosure.

FIG. 3 is a block diagram illustrating a system for processing health balance information using a scale device for generating a balance profile for a patient according to some embodiments of the disclosure.

FIGS. 4A and 4B illustrate a report of a patient's balance profile according to some embodiments of the disclosure.

FIG. 5 is a block diagram illustrating a patient's balance profile according to some embodiments of the disclosure.

FIG. 6 is a screen illustration of an example profile showing earned medals in a patient's balance profile according to some embodiments of the disclosure.

FIG. 7 is a screen illustration of an example report showing population balance trends according to some embodiments of the disclosure.

FIG. 8 is a screen illustration of an example balance assessment for a patient's balance profile according to some embodiments of the disclosure.

FIG. 9 is another screen illustration of an example balance assessment for a patient's balance profile according to some embodiments of the disclosure.

### DETAILED DESCRIPTION OF SOME EMBODIMENTS

A system, according to some embodiments of this disclosure, collects balance and health information from one or more devices according to one or more methods. The balance and health information may include a balance profile, electronic health record information, electronic medical record information, telehealth information, environment or location data. Additional health and activity information could be collected from an activity tracker, such as a pedometer, wrist-worn device, clothing embedded device, or any alternative body-worn or carried device. Wearable health sensors, which collect information such as heartrate, blood pressure, blood oxygen, sleep analysis, or any variety of medical vitals may also contribute to the health information of an individual. Further information may be collected from devices, such as a balance platform, force plate, insoles, weight scale, mat, floor panel, shoe, sock, walker, cane, prosthetic or robotic leg, etc. for the purpose as a balance measurement device or postural stability measurement device, for example. Health and balance information may also be collected from fall prediction devices, such as activity trackers, smart watches, medical alert systems, or camera-based systems. Still further information may be collected from sensor-enabled mobility and home devices, such as walkers, canes, wheelchairs, smart beds, toilets, chairs, and couches, which may assist one's lifestyle or accessibility.

Additional information may be collected in the natural environment or in a healthcare setting, in-person, or virtual. Such information may be collected from an in-patient clinic, outpatient clinic, hospital, emergency clinic, acute care center, ambulatory care center, physical therapy clinic, chiropractor's office, movement disorders clinic, and/or home or public space, for example. Other methods and embodiments include using the balance scale for monitoring of a room or space via camera and/or microphone. The scale could also interact with people in the environment via Bluetooth, audio output, visuals displayed on the scale, physical input, etc. The balance health information is used in conjunction with this information collected in the natural environment or healthcare setting in order to assess overall balance health of an individual.

A machine learning algorithm may be used, in some embodiments of the disclosure, for determining balance health assessment risk score based on data acquired from a variety of inputs according to some embodiments of the disclosure. Strategies for determining the final balance health assessment risk score include principal component analysis, Bayesian classification, neural network or deep-learning based strategies, SVMs (support vector machines), autoencoder algorithms, CNNs (convolutional neural networks), variational autoencoders, and/or supervised and unsupervised learning approaches more broadly. In addition to stability metrics, raw data may also be provided to the artificial intelligence. In the case of a neural network, the network may be trained (using training data from individuals with a known fall history) to identify combinations of metrics and raw data indicative of fall risk. Any combination of the data described in the examples below may be used to train a supervised model and that model used to determine fall risk for individuals.

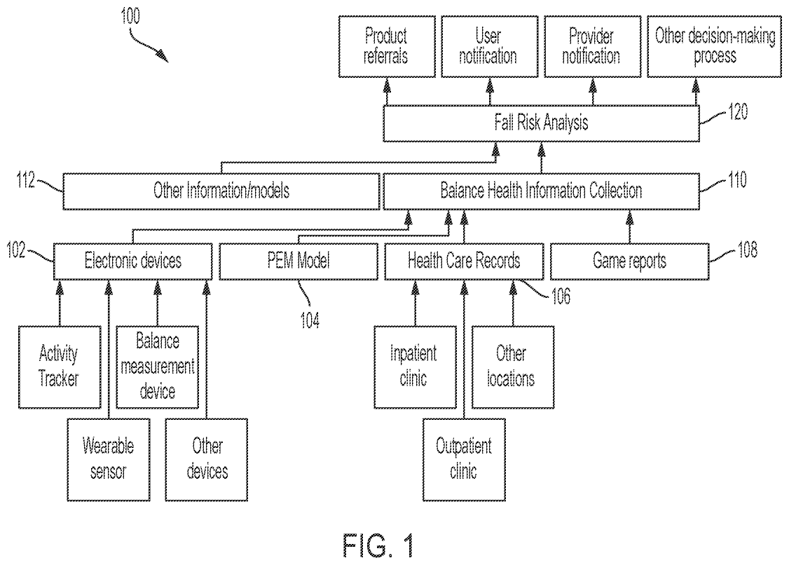

FIG. 1 is a block diagram illustrating a system for processing health balance information according to some aspects of the disclosure. A system 100 may collect balance health information 110 and use the information to perform a fall risk analysis 120, wherein the balance health information 110 may be collected from one or more sources. Balance health information 110 may comprise information from electronic devices 102 associated with the user or a third-party monitoring person. For example, collected information may include data from electronic devices 102, which may include environment or location data, data from activity trackers (e.g., pedometer, wrist worn, body worn, clothing embedded), data from wearable health sensors (e.g., heartrate, blood pressure, blood oxygen etc.), data from a balance measurement device (e.g., balance platform, force plate, insoles), data from postural stability measurement devices (e.g., force plate, insole, weight scale), data from a fall prediction device (e.g., activity tracker, apple watch, life alert, camera based system), data from a sensor enabled mobility device (e.g., walker, cane, wheelchair), data from a home sensor (e.g., smart bed, toilet, chairs, couches), or other devices.

Balance health information may further comprise data from a postural state analysis and/or a punctuated equilibrium model (PEM) 104 for the patient. Static and dynamic postural states facilitate a punctuated equilibrium model (PEM) 104 of postural stability. The PEM is defined as periods of stability punctuated by dynamic trajectories. Alerting a person to that transient dynamic and thereby dangerous state can help them take instant action to avoid the imminent fall. Base measures of postural instability from the PEM 104 are identified as: number of equilibria, equilibria dwell time, and size of equilibria. The number of equilibria may include a number of equilibria identified in a time series. The dwell time may include a size of a pentagon or other shape that represents the time spent in that particular equilibrium. The size of equilibria may include an average (or other characteristic such as mean, maximum, or minimum) of each point in the equilibrium to the center of the corresponding equilibrium. Data 104 may be generated as described in U.S. Pat. No. 10,307,084, which is incorporated by reference herein. Although the base punctuated equilibrium model (PEM) stability metrics may be sufficient for determining postural states, additional stability metrics may improve determination of postural states and/or allow for the determination of fall risk and/or classifying an individual's fall risk.

The PEM data 104 may be an output of a machine learning algorithm operating on one or more of the data described herein to evaluate a person's balance. PEM data 104 may be generated using a machine learning algorithm, such as to classify dynamic and static postural states for a PEM with HMM techniques, using advanced PEM stability metrics, and COP (center of pressure) data according to some embodiments of the disclosure. After acquiring appropriate data over a period of time, machine learning algorithms may receive the COP data and calculate, for example, postural states. The period of time may be predetermined by the patient, healthcare provider, and/or a third-party monitoring person. Then, the machine learning algorithms may be used to estimate fall risk and/or classify fall risk based on those postural states. In some embodiments, the machine learning algorithms may be used to classify postural states for calculating subsequent metrics and determine fall risk thresholds. In other embodiments, the machine learning algorithm may be used to classify fall risk as the objective function, either with or without the preceding determination of postural states. In some embodiments, the estimated fall risk may also be based, in part, on at least one of clinical records, exercise, lifestyle inputs, weight, body fat composition, body mass index, level of hydration, medication consumption, alcohol consumption, sleep, steps per day, exercise, time spent sitting, and/or strength.

A complete balance profile may be created for each user, and the profile may identify health areas that may be affecting the patient's balance. The balance profile may include and/or be based on some or all of collected information 110. Each balance health information 110 may be individually weighted when calculating a balance health assessment risk score or determining the patient's balance profile. There are a number of advantages of the PEM approach. Firstly, the technique classifies otherwise uniform data, identifying stable regions and dynamic trajectories, with the latter being viewed as unstable. Threshold functions are described to identify the postural state users are in, whether for real-time identification or long-term detection of postural instability. Further, the approach creates relative measures of stability that create independence from height and weight, location of the feet, or known stability boundaries.

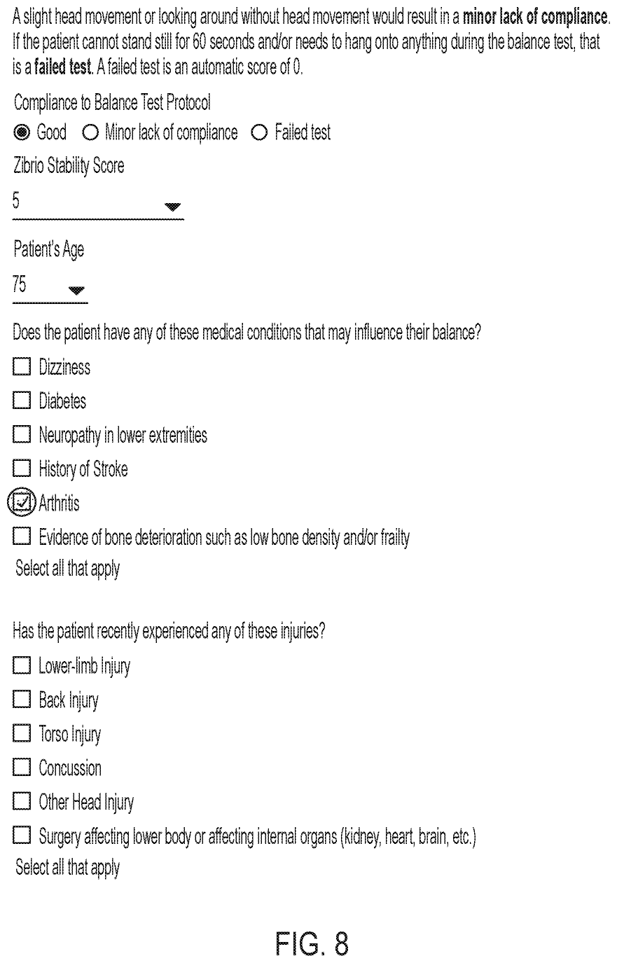

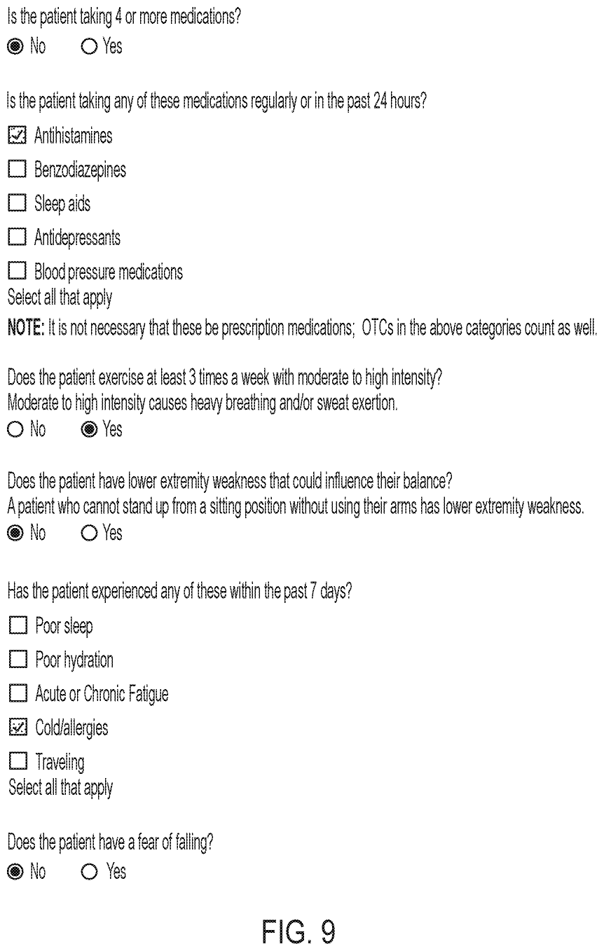

The balance health information 110 may also be collected from health care records 106. For example, collected information may include data 106 from one or more of individual health information (balance profile, balance care inputs), electronic health record information (EHRs), electronic medical record information (EMRs), in person evaluations, telehealth evaluations, and/or records from other locations. The data 106 may be collected from different locations, such as an in-patient clinic, outpatient clinic, hospital, acute care center, ambulatory care center, physical therapy clinic, chiropractor's office, movement disorders clinic, and/or home or public space. Other data 106 may be collected from a history of falls or health assessment questionnaire (e.g., the examples shown in FIG. 8 and FIG. 9), Johns Hopkins Fall Risk Assessment Tool (JHFRAT), the CDC's Stopping Elderly Accidents, Deaths, and Injuries (STEADI) initiative, Timed Up and Go test, and the Tinetti Performance-Oriented Mobility Assessment (POMA). There is also a variety of potential data 106 from clinical fall risk assessment tools, including, but not limited to, the 30 second chair stand test, four-stage balance test, ten meter walk test (10MWT), Berg Balance Scale, Falls Efficacy Scale International (FES-I), Functional Gait Assessment (FGA), Single-leg Stance (SLS) test, multidirectional reach test (MDRT), and the Fullerton Advanced Balance (FAB) Scale.

Balance health information 110 may also include game reports 108 from applications, such as game applications, related to balance training and/or exercise training that test and quantify a subject's balance health. The game reports 108 may provide real-world and/or instantaneous information regarding the subject. In some embodiments, the game application may also interact with physiological metrics and other data contained in the other collected information 110. For example, input to the game application or other software may include data from an activity tracker (wrist-worn, body-worn, or clothing-embedded pedometer), a wearable health sensor (heartrate, blood pressure, blood oxygen, etc.), a balance measurement device (balance platform, force plate, insoles), a postural stability measurement device (force plate, insole, weight scale), a fall prediction device (activity tracker, apple watch, life alert, camera based system), a sensor-enabled mobility device (walker, cane, wheelchair), and/or a sensor-enabled home (smart bed, toilet, chairs, couches). In some embodiments, the fall risk analysis 120 may also interact with the system determining the PEM. For example, the measured center of pressure may be input to a software interface and/or the measured center of pressure may be used as a control for an interactive game application. The balancing training and/or exercise training may be provided as a score which can be tracked and shared as an electronic communication through social media and/or smartphone applications.

According to certain embodiments, such balance health information 110 may be further used as inputs in generated a patient-specific chatbot that is designed to convey the results of the patient's fall risk analysis 120. This may be an interactive experience for the patient using artificial intelligence to generate advice based, at least in part, on the input from the patient's electronic devices, PEM model, healthcare records, game reports, and/or balance health information. The chatbot may be able to answer questions input from the patient before and/or after a fall risk analysis report has been generated. This patient-specific chatbot may be implemented into an application to be executed on the patient's electronic device(s).

Information collected from the one or more sources contribute to the balance health information 110 which is used to determine the fall risk analysis 120. The fall risk analysis 120 may also consider other information and/or models 112. Information 112 may include user input to prompts in applications, such as answers to a questionnaire presented on the user's mobile device. For example, fall risk analysis 120 may operate on the collected balance health information 110 using a trained machine learning (ML) algorithm, in which the model is received as other information 112. The balance health information 110 may be individually weighted when calculating the fall risk analysis 120. The variables considered, such as balance health information 110 and/or other information or models 112, when calculating the fall risk analysis help to identify the contributors to fall risk and provide personalized solutions for the patient to decrease the fall risk and improve fall risk prediction.

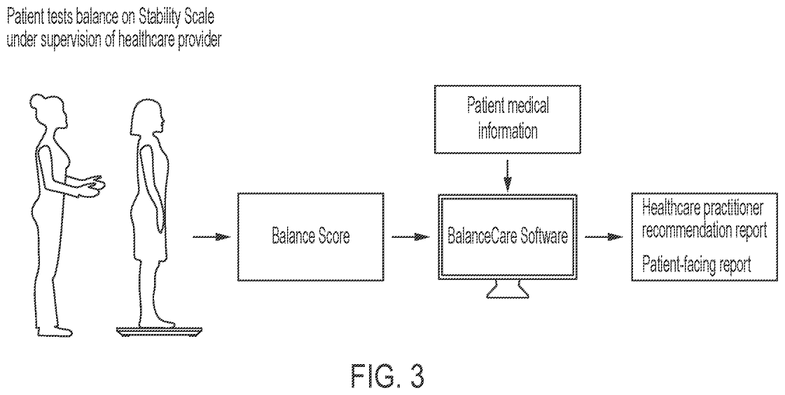

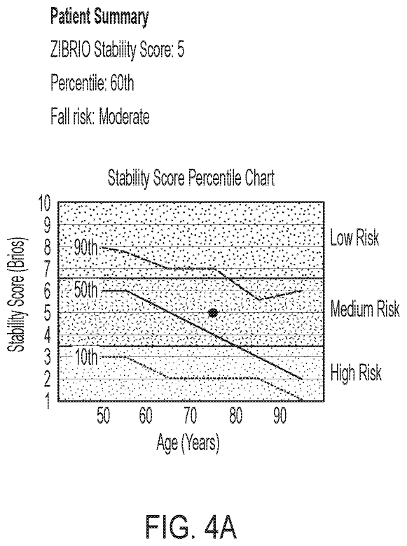

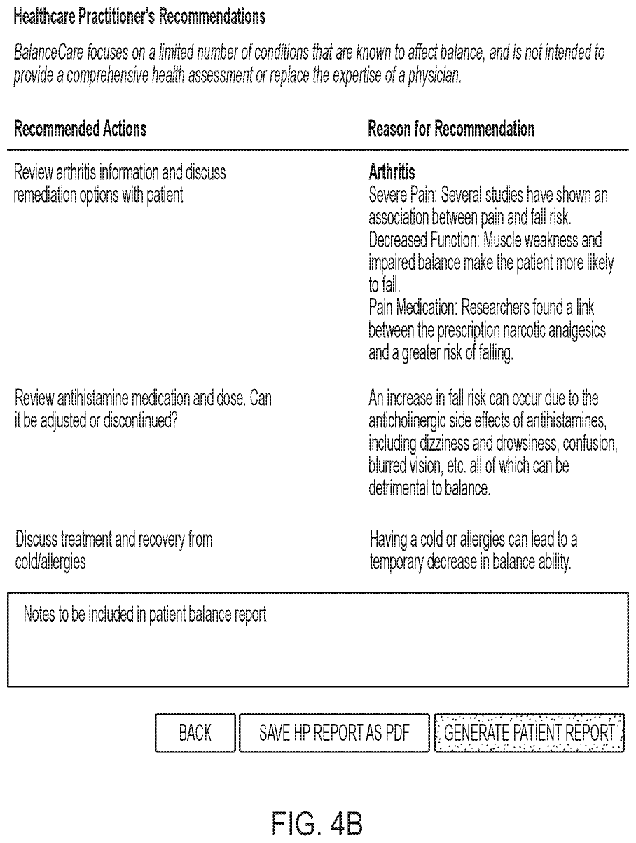

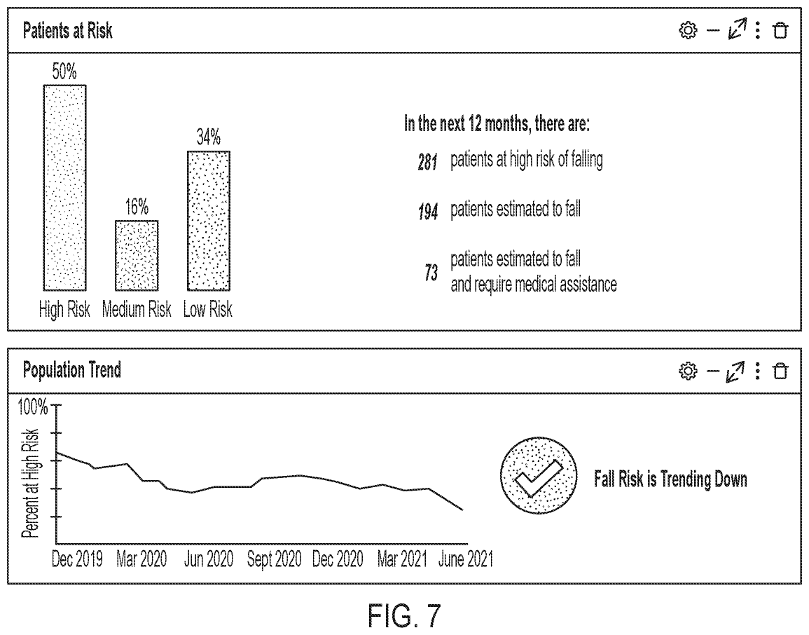

The fall risk analysis 120 may produce one or more outputs, such as a product referral, a user notification, a provider notification, and/or other data to assist other decision-making processes. FIG. 3 is a block diagram illustrating one specific embodiment of the system shown in FIG. 1 using a scale device for generating a balance profile for a patient based, at least in part, on a balance score, patient medical information, and/or other balance health information or models to determine a healthcare practitioner recommendation report and a patient-facing report. FIGS. 4A-B is an example report describing the patient's balance profile, including fall risk, that may be determined using fall risk analysis 120. In some embodiments, the output of the fall risk analysis 120 may be provided to a provider for a plurality of patients, such as shown in the example of FIG. 7. The fall risk analysis 120 for a plurality of patients may include categorized values of patients based, at least in part, on their fall risk or fall likelihood, and/or a population trend of the fall risk for the plurality of patients as illustrated in FIG. 7.

In some examples, the machine learning algorithm is selected from the group consisting of a deep learning algorithm, a neural network algorithm, an artificial neural network algorithm, a supervised machine learning algorithm, a linear discriminant analysis algorithm, a quadratic discriminant analysis algorithm, a support vector machine algorithm, a linear basis function kernel support vector algorithm, a radial basis function kernel support vector algorithm, a random forest algorithm, a genetic algorithm, a nearest neighbor algorithm, k-nearest neighbors, a naive Bayes classifier algorithm, a logistic regression algorithm, or a combination thereof.

In some examples, including any of the foregoing, the trained model is used to treat a patient having balance issues or a high fall risk. In some examples, the trained model is used to identify activities, postures, or other states associated with fall risks, and used to treat a patient. In some of these examples, the trained model uses fall risks observed by analyzing information from sample data of patients known to have high fall risk, such that other subject patients with similar balance profiles may be identified. In some embodiments, one output of the fall risk analysis 120 may be a personalized digital model of the patient's postural control. The one or more outputs of the fall risk analysis 120 may analyze and identify the major and minor contributors and/or factors which may affect a patient's balance. For example, such contributors may include the medications taken by the patient and/or at least one of the plurality of variables used to calculate the fall risk analysis 120. Using such sample data of patients known to have high fall risk, the output of the fall risk analysis 120 may further include a digital model of postural control across populations. The fall risk analysis 120 may further determine and identify healthcare targets of care for optimization of fall prevention outcomes. For example, it may find correlations between balance and/or fall risk and a medication taken by patients with one or more diagnosed or undiagnosed health conditions. Further, the fall risk analysis 120 may find a different correlation between balance and/or fall risk and the medication with a different condition. The correlation may be represented by a percentage indicating the level of influence or another variable which may quantify the effects on the patient's balance.

Output of the fall risk analysis 120 may be accessible by the patient and/or a third-party monitoring person in an effort to improve fall risk prediction and take preventative measures. This may also assist healthcare providers in prescribing medications or activities that may influence a person's balance or fall risk. Output from the fall risk analysis 120 may also assist in recognizing and identifying early onset health conditions such as, for example, Parkinson's, Multiple Sclerosis, incontinence, stroke, spinal injury, dementias, neuropathy, osteoporosis, or any other health condition, based, at least in part, on data collected from a plurality of patients, the patient's healthcare records, and/or a game report of the patient. The output of the fall risk analysis 120 also may determine a risk for health conditions that are yet to be diagnosed to the patient. The output may include an optimized care plan that uses the plurality of inputs to determine a prioritized care plan specific to the user. The care plan may target a specific health outcome, such as falling or any diagnosable condition, or may be for the betterment of the patient's health, balance, and/or fall risk. The output may further include optimized medication routines, medication brand(s), dosage recommendations, and/or medication adjustment recommendations, which may include the adding or removing of a medication from a patient's routine, or raising or lowering the dosage prescribed. The output may further include optimized or recommended nutrition plans, such as dietary restrictions or additives, supplements, medications, etc. House plans, house designs, or interior design layouts may also be recommended to the user, such as wider doorways or hallways for wheelchair or walker accessibility, or recommendations for safety rails or grab bars for those at higher risk of falling located anywhere in the home. Recommendations to the user may further include exercise routines, transport choice, vehicle choice, bed or chair choice, vacation/activity/tour opportunities or recommendations, and/or access to wellness services for a longer and healthier life, all of which may provide proper support for the patient's optimized lifestyle. Any of these recommendations may be based, at least in part, on the inputs used to calculate the fall risk analysis 120 and/or sample data of patients known to have high fall risk. These recommendations may be delivered to the patient or third-party monitoring person through the same means as the output of the fall risk analysis 120.

The output of the fall risk analysis 120 may be output to a user, provided to another system, and/or provided to another process for further data generation and/or decision-making. For example, another process may determine a fall risk score based on the fall risk analysis 120. The risk score and/or alerts regarding the risk score may be provided through an electronic communication device (e.g., email, text message, voice), a software interface (e.g., smartphone app, pc software, web user interface), a smart home technology (e.g., smart speaker, internet connected lights, outlets or other internet of things devices wirelessly or directly connected), a patient monitoring interface (e.g., smartphone app, pc software, web user interface), a hospital medical device (e.g., smart bed, monitoring system, electronic patient chart), and/or a network-enabled wearable device (e.g., fall alert pin, broach, article of clothing (shirt, socks, shoes).

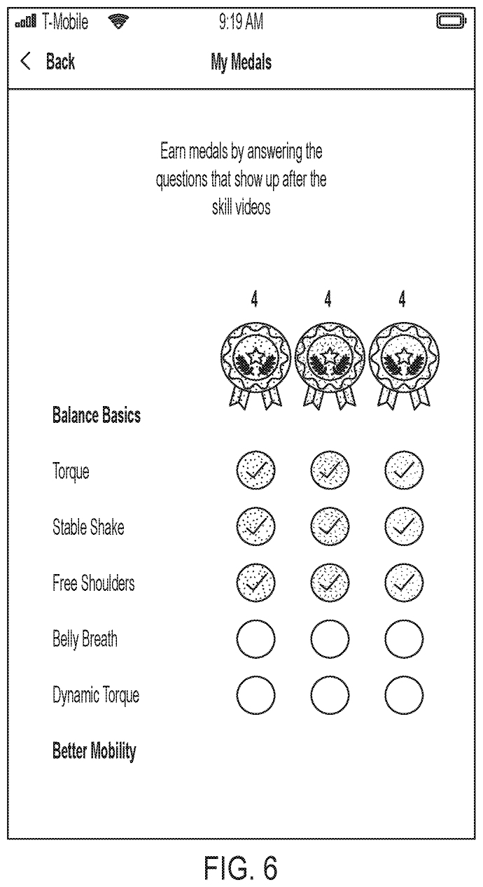

In some embodiments, the fall risk analysis 120 may be used to determine additional operations to be performed by the user. For example, the fall risk analysis may be used to determine an exercise training progression (including a next game application or level in the game to present to the subject), to determine a balance training progression (including a next game application or level in the game to present to the subject), and/or to determine an avatar expression (to be displayed in the game application and/or output to social networks). The applications may use balance data in conjunction with other inputs to provide clinical recommendations, as well as provide programs for balance basics, better mobility, better feet, head balance, and daily goals, for example. To promote gamification and positive reinforcement of participation in these games and training activities, the user may earn medals by answering questions that populate after the skill videos. Using the patient's balance profile and training progression, the software may recommend a marketplace of products. An example of a profile showing earned medals is shown in FIG. 6.

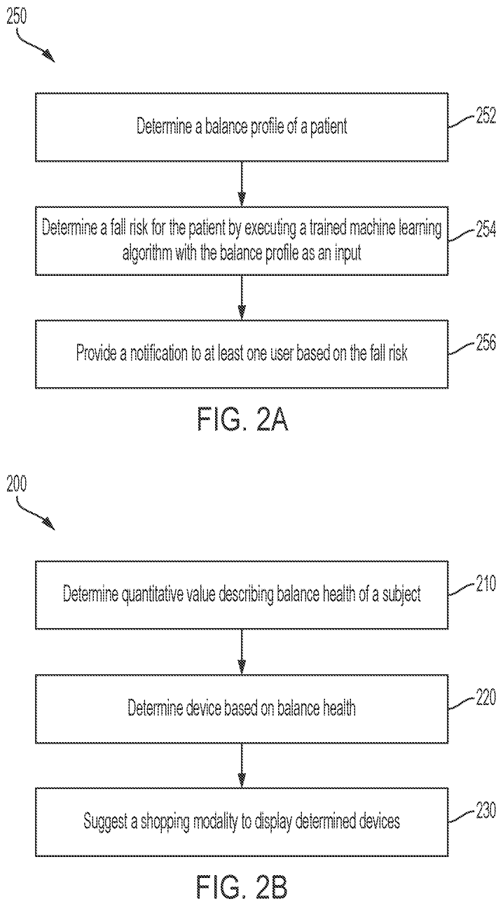

One method of determining a fall risk based on at least some of the processing described with reference to FIG. 1 is shown in FIG. 2A. A method 250 includes, at block 252, determining a balance profile of a patient. The balance profile may be determined from the balance health information collection 110 and/or other information/models 112. In some embodiments, the balance profile includes information from a postural state analysis and/or PEM model 104, which may include information regarding postural states of the user determined from devices such as the electronic devices 102.

At block 254, the method determines a fall risk for the patient by executing a trained machine learning algorithm with the balance profile as an input. Fall risk analysis 120 may be performed based on the balance profile comprising the health information collection 110 and/or other information/models 112.

At block 256, the method provides a notification to at least one user based on the fall risk. The notification may include one of a product referral, user notification (e.g., to a patient or patient's relative), and/or a provider notification.

The fall risk analysis 120 may be used to recommend products to the subject user. The products may include balance/exercise training delivered through an application on an electronic device. The products may include recommendations modified with physiologic metrics, and suggest balances and/or wearables from a marketplace of products. The marketplace may include a listing of products or services and associated conditions, scores, data, and/or other conditions, criteria, or rules. For example, a marketplace participant may list their product/service along with a condition of a fall risk score between a first threshold and a second threshold. A method of recommending a product or service is illustrated in FIGS. 2A and 2B. The method 200 includes determining a quantitative value describing a balance health of a subject user at block 210. At block 220, a device is determined to match one or more criteria regarding the quantitative value and/or other data regarding the subject user. At block 230, a shopping modality is suggested to display the determined devices. For example, an alert may be pushed to the subject user's devices with a link to a marketplace, and, when the user loads the marketplace, the determined device of block 220 is presented.

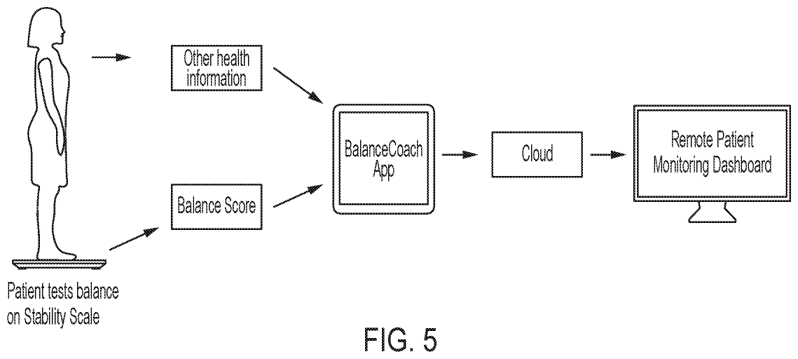

The fall risk analysis 120 may output analysis, such as fall risk scores, to a remote service for monitoring. For example, the output may be provided to software that remotely monitors balance/fall risk and connects to cloud and/or electronic medical records to deliver results of the analysis. One embodiment of remote monitoring is shown in FIG. 5.

In certain embodiments, the fall risk analysis 120 may include deep learning algorithms for diagnosis of poor balance, fall risk, ataxia, and other physiological and activity states. These can include autoencoder algorithms, convolutional neural networks, variational autoencoders, and other approaches. These methods may include stabilograms, computerized dynamic posturography, sway referencing, and/or postural sway data. These can include methods that use medical records and methods that do not.

Example uses of electronic devices according to the embodiments described above may include one or more of: using a scale for monitoring of a room or space, via camera or via microphone; using the scale to interact with people in the house via Bluetooth, via audio speaker, and/or via visuals on the scale; and/or use a scale to communicate with people outside of the house via Voice over IP (VoIP), via email, via the world-wide-web, and/or via peer-to-peer network.

Electronic devices as described herein can be any variety of a mobile device, smartwatch, smartphone, tablet, computer, cloud-based service and/or data analysis module. If the electronic communication device is a tablet, the user may hold the device or have it near the scale during the test or attached to a wall in front of the user. If the electronic communication device is a smartphone, the user may hold the device or have it near the scale during the test or attached to a wall in front of the user. The electronic communication device may transmit and receive data over any type of communications link, such as email, text message, voice message, etc.

In one embodiment, the electronic communication device may comprise one or more integrated circuits (e.g., microcontroller, etc.) and/or discrete components on a printed circuit board or other electronic packaging technology. For example, the electronic communication device may include a RF transceiver for transmitting and/or receiving data prepared by the signal preparation module. The electronic communication device may transmit and receive data over any type of communications link, for example, the electronic communication device may include a wireless transceiver utilizing an RF network such as a Bluetooth network. The electronic communication device may include authentication capability to limit transfer of data to only authorized devices. Additionally, the electronic communication device may encrypt data before transmission to prevent unauthorized access to the information. In some embodiments, the electronic communication device may include a smartphone, smartwatch, tablet, or laptop that includes the ICs, components, and/or code described above.

A software interface used to alert a user, care provider, or any other intended recipient may comprise a smartphone application, PC software, a web user interface, or any other software interface with may utilize any type of communications link. Smart home technology including, but not limited to, a smart speaker, internet connected lights or appliances, outlets, or other internet of things devices wirelessly or directly connected, may be used to communicate balance health assessment risk scores and associated data. Patient monitoring interfaces or hospital medical devices may include smartphone applications, PC software, a web user interface, smart bed, monitoring system, electronic patient card, or any other device which may be used to monitor user health or balance information. Bluetooth enabled wearable devices, either worn or carried by the individual, include technologies such as fall alert pins, broaches, articles of clothing, smartwatches, Fitbits, etc.

The disclosed system is further able to provide clinical decision-making support based on balance health assessment risk via an individual's patient chart, electronic health or medical record, electronic communication device, software interface, smart home technology, patient monitoring interface, hospital medical device, and/or Bluetooth enabled wearable device.

The disclosed system is further able to provide software as a medical device based on balance health assessment risk via an individual's patient chart, electronic health or medical record, electronic communication device, software interface, smart home technology, patient monitoring interface, hospital medical device, and/or Bluetooth enabled wearable device.

One embodiment of the disclosure may include two or more load sensors that collect load data for a period of time. The system may also include a signal preparation module housed within a balance device with wireless transmission capability for transmitting the load data to an electronic communication device and, according to one aspect of this disclosure, then to a cloud-based data analysis module. The signal preparation module may contain analog-to-digital converters (ADCs), timers, and other discrete or integrated components used to convert the output of the load sensor module(s) to digital data values. The signal preparation module may include any general-purpose processor, a microprocessor, amplifier, other suitably configured discrete or integrated circuit elements, and memory. The memory may be any type of volatile or non-volatile storage medium including solid-state devices such as DRAM, SRAM, FLASH, MRAM or similar components for data storage. The signal preparation module may be configured with circuitry and/or instructions to process data from the load sensors (e.g., convert analog to digital or otherwise interpret the load sensor signals) and/or package the data for transmission over a network connection or other bus (either wired or wireless), such as by forming packets or frames for network transmission or assembling data for USB transfer. A power source such as a battery may be attached by any suitable arrangement for providing power to the circuits of the load detecting module and signal preparation module.

The use of physiological metrics as input to the software interface include data from any activity tracker, wearable health sensor, balance measurement device, postural stability measurement device, sensor enabled mobility device, or sensor enabled home device or appliance. The game score is used to determine training progression in balance and/or exercise, which helps to identify the next game based on previous performance and allows the user to improve performance by increasing his or her game score. The game score may also be used to inform avatar creation and expression, which may be tracked and shared as electronic communication via social media or any communications link. In some embodiments, the next game may be based, at least in part, on previous game performance, recommendations from a third-party monitoring person, incremental difficulty from a previous game, and/or recommendations from other users via social media or any communications link.

Although the present disclosure and certain representative advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. For example, although processors are described throughout the detailed description, aspects of the invention may be executed by any type of processor, including graphics processing units (GPUs), central processing units (CPUs), digital signal processors (DSPs), application-specific integrated circuits (ASICs), and/or other circuitry configured to execute firmware or software that executes the instructions and methods described above. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method, comprising:
receiving a postural state analysis;
receiving at least one healthcare record for a patient;
receiving at least one game report for the patient;
determining a fall risk for the patient by executing a first machine learning algorithm with the postural state analysis, the at least one healthcare record for the patient, and the at least one game report for the patient as inputs; and
providing a notification to at least one user based on the fall risk, wherein providing the notification to the at least one user is based on the fall risk satisfying one or more criteria.

2. The method of claim 1, wherein the postural state analysis is based on a second machine learning algorithm different from the first machine learning algorithm.

3. The method of claim 2, wherein the postural state analysis is based on a punctuated equilibrium model (PEM) for the patient.

4. The method of claim 1, wherein the notification identifies at least one factor contributing to the fall risk of the patient.

5. The method of claim 1, further comprising providing a medication adjustment recommendation to a healthcare provider of the patient.

6. The method of claim 1, further comprising providing a product referral based on the fall risk satisfying one or more criteria.

7. An information handling system, comprising:
a memory;
a processor coupled to the memory, wherein the processor is configured to perform steps comprising:
receiving at least one healthcare record for a patient;
receiving at least one game report for the patient;
determining a fall risk for the patient by executing a first trained machine learning algorithm with the postural state analysis, the at least one healthcare record for the patient, and the at least one game report for the patient as inputs; and
providing a notification to at least one user based on the fall risk, wherein providing the notification to the at least one user is based on the fall risk satisfying one or more criteria.

8. The information handling system of claim 7, wherein the postural state analysis is based on a second machine learning algorithm different from the first machine learning algorithm.

9. The information handling system of claim 8, wherein the postural state analysis is based on a punctuated equilibrium model (PEM) for the patient.

10. The information handling system of claim 7, wherein the notification identifies at least one factor contributing to the fall risk of the patient.

11. The information handling system of claim 7, further comprising providing a medication adjustment recommendation to a healthcare provider of the patient.

12. The information handling system of claim 7, further comprising providing a product referral based on the fall risk satisfying one or more criteria.

13. A computer program product, comprising:
a non-transitory computer readable medium comprising code for performing steps comprising:
receiving at least one healthcare record for a patient;
receiving at least one game report for the patient;
determining a fall risk for the patient by executing a trained machine learning algorithm with the postural state analysis, the at least one healthcare record for the patient, and the at least one game report for the patient as inputs; and
providing a notification to at least one user based on the fall risk, wherein providing the notification to the at least one user is based on the fall risk satisfying one or more criteria.

14. The computer program product of claim 13, wherein the postural state analysis is based on a second machine learning algorithm different from the first machine learning algorithm.

15. The computer program product of claim 14, wherein the postural state analysis is based on a punctuated equilibrium model (PEM) for the patient.

16. The computer program product of claim 13, wherein the notification identifies at least one factor contributing to the fall risk of the patient.

17. The computer program product of claim 13, further comprising providing a medication adjustment recommendation to a healthcare provider of the patient.

18. The computer program product of claim 13, further comprising providing a product referral based on the fall risk satisfying one or more criteria.
